SEQUENCE LISTING (11 sequences)

SEQ ID NO: 1 - DNA, 51 bp, Artificial Sequence
  Synthetic
  misc_feature (34)..(39): n is a, c, g, or t
gatcggaagagcacacgtctgaactccagtcacnnnnnnatctcgtatgcc

SEQ ID NO: 2 - DNA, 58 bp, Artificial Sequence
  Synthetic
aatgatacggcgaccaccgagatctacactctttccctacacgacgctcttccgatct

SEQ ID NO: 3 - DNA, 21 bp, Artificial Sequence
  Synthetic
acggcgaccaccgagatctac

SEQ ID NO: 4 - DNA, 193 bp, Artificial Sequence
  Synthetic
caagcagaagacggcatacgagatcgttaggatgtgactggagttcagacgtgtgctcttccgatctagattctggcgggtgctgatagtgtatcctactacttttgacttctctgtagaggggagtctcagctagatcggaagagcgtcgtgtagggaaagagtgtagatctcggtggtcgccgtatcatta

SEQ ID NO: 5 - DNA, 37 bp, Artificial Sequence
  Synthetic
gatctacactctttccctacacgacgctcttccgatc

SEQ ID NO: 6 - DNA, 37 bp, Artificial Sequence; identical to SEQ ID NO: 5
  Synthetic

SEQ ID NO: 7 - DNA, 25 bp, Artificial Sequence
  Synthetic
cggaagagcacacgtctgaactcca

SEQ ID NO: 8 - DNA, 37 bp, Artificial Sequence; identical to SEQ ID NO: 5
  Synthetic

SEQ ID NO: 9 - DNA, 25 bp, Artificial Sequence
  Synthetic
cggaagagcacacgtctgaactcca

SEQ ID NO: 10 - DNA, 37 bp, Artificial Sequence; identical to SEQ ID NO: 5
  Synthetic

SEQ ID NO: 11 - DNA, 25 bp, Artificial Sequence
  Synthetic
cggaagagcacacgtctgaactcca

(19) United States
(12) Patent Application Publication
(10) Pub. No.: US 2021/0017596 A1
(43) Pub. Date: Jan. 21, 2021

(54) SEQUENTIAL SEQUENCING METHODS AND COMPOSITIONS

(71) Applicant: Qiagen Sciences, LLC, Germantown, MD (US)

(72) Inventors: Yanhong Tong, Boxford, MA (US); Seiyu Hosono, Stoneham, MA (US); Reto Mueller, Brookline, MA (US); Thomas Perroud, Lexington, MA (US)

(21) Appl. No.: 16/867,680

(22) Filed: May 6, 2020

Related U.S. Application Data
(60) Provisional application No. 62/845,033, filed on May 8, 2019.

(51) Int. Cl. C12Q 1/6874; C12N 15/10

(52) U.S. Cl. CPC C12Q 1/6874; C12N 15/1093

(57) ABSTRACT

This invention relates in general to methods of sequencing multiple distinct and separate polynucleotide fragments and regions in a sequential order, such as on a flow cell surface. The invention provides methods that solve prior art problems with regard to sequential sequencing, and that provide advantages including low cost, shorter turn-around-time, high efficiency, and easy implementation.

### FIELD OF THE INVENTION

This invention relates in general to methods of sequencing multiple distinct and separate polynucleotide fragments and regions in a sequential order, such as on a flow cell surface. The invention provides methods that solve prior art problems with regard to sequential sequencing, and that provide advantages including low cost, shorter turn-around-time, high efficiency, and easy implementation.

### BACKGROUND OF THE INVENTION

The libraries prepared for next generation sequencing (NGS) technology always include multiple distinct and separate segments to be sequenced. These segments can be insert sequences from sample (regions of interest), index sequences, and/or barcode sequences. To sequence each segment, more than one sequencing primers are included for overall sequencing processes on a flow cell in a sequential manner. For each sequencing segment, a specific sequencing primer binds, i.e., hybridizes, to the upstream adjacent position of the target sequences and the corresponding DNA segment is sequenced. This process is repeated in a sequential manner if another separate segment is to be sequenced.

Rolony, which is clonally amplified through RCA process (rolling circle amplification), is one template used in the prior art for sequencing. However, there are limited approaches to perform a sequential sequencing process as described above. An existing approach is to perform the sequencing of the second block by blocking all SBS enzymatic activity from the first segment by the addition and incorporation of dideoxy-dNTP with a DNA polymerase. However, this approach can be cost prohibitory due to the use of large amounts of enzymes and dideoxy dNTP, especially when there is a large dead volume required for the sequencing instrument.

Thus, what is needed are methods that provide solutions for sequential sequencing, such as sequencing on rolony.

### SUMMARY

The invention provides methods that solve prior art problems with regard to sequential sequencing of a polynucleotide template, exemplified by, but not limited to, sequencing on rolony. The invention's methods provide advantages including low cost, shorter turn-around-time, high efficiency, and easy implementation. The invention's description herein is not intended to describe each disclosed embodiment or every implementation of the present invention. The invention's description herein exemplifies some illustrative embodiments. In several places throughout the invention's description herein, including the drawings, guidance is provided through exemplary embodiments, which can be used in various combinations. In each instance, the exemplary embodiments serve only as a representative group and should not be interpreted as exclusive and/or limiting embodiments.

In one embodiment, the invention provides a method for sequentially sequencing a plurality of target sequences, said method comprising
i) providing
   a) a single-stranded DNA sequence comprising a plurality of target sequences, wherein each member of said plurality of target sequences is operably fused to an oligonucleotide sequence, and wherein the sequence of said oligonucleotide sequence fused to each said member of said plurality of target sequences is different from the sequence of said oligonucleotide sequence fused to each of the other members of said plurality of target sequences, and
   b) a plurality of sequencing primers, wherein each member of said plurality of sequencing primers is complementary to and specifically bind with a different said oligonucleotide sequence,
ii) sequentially sequencing two or more said member of said plurality of target sequences, said sequentially sequencing comprises
   a) hybridizing one or more first member of said plurality of sequencing primers to its said complementary oligonucleotide sequence in a first reaction mixture to produce a first hybridized sequencing primer,
   b) extending said first hybridized sequencing primer to produce a first sequencing read, said extending comprises producing a first double-stranded DNA sequence containing a complement of a first said member of said plurality of target sequences operably fused to said one or more first member of said plurality of sequencing primers, and comprises sequencing at least one strand of said first double-stranded DNA sequence,
   c) removing the extended sequence produced in step b) from said first reaction mixture,
   d) hybridizing one or more second member of said plurality of sequencing primers to its said complementary oligonucleotide sequence in a second reaction mixture to produce a second hybridized sequencing primer, and
   e) extending said second hybridized sequencing primer to produce a second sequencing read, said extending comprises producing a second double-stranded DNA sequence containing a complement of a second said member of said plurality of target sequences operably fused to said one or more second member of said plurality of sequencing primers, and comprises sequencing at least one strand of said second double-stranded DNA sequence.
      In one embodiment, the method further comprises repeating steps c) to e) to sequence members of said plurality of target sequences that are different from both said one or more first member of said target sequence and said one or more second member of said target sequence. In one embodiment, the method lacks using a blocking reagent. In one embodiment, the method lacks addition of one or more said blocking reagent to both said first and second reaction mixtures. In one embodiment, said method lacks incorporation of one or more said blocking reagent into any of said first double-stranded DNA sequence produced in step b) and of said second double-stranded DNA sequence produced in step e). In one embodiment, said removing of step c) comprises washing with a buffer having a temperature higher than a melting temperature of said first double-stranded DNA sequence produced in step b). In one embodiment, said removing of step c) comprises washing with a buffer having low ionic strength. In one embodiment, said removing of step c) comprises washing with a buffer comprising proteins having 3′ to 5′ exonuclease activity, and/or enzyme having 5′ to 3′ exonuclease activity, and/or compound that denatures DNA. In one embodiment, said removing of step c) comprises washing with a buffer comprising a compound that denatures DNA, or reducing melting temperature of double-stranded DNA. In one embodiment, said compound that denatures DNA comprises one or more of sodium hydroxide, formamide, and betaine. In one embodiment, said double-stranded DNA sequence comprises at least a portion of a rolony. In one embodiment, said double-stranded DNA sequence comprises the extended sequencing segment. In one embodiment, said sequencing steps b) and e) comprises at least two sequencing cycles. In one embodiment, the number of said sequencing cycles of steps b) and e) is different. In one embodiment, said first target sequence is shorter than said second target sequence, and said number of said sequencing cycles of step b) is less (i.e., lower) than step e). In one embodiment, said first target sequence is longer than said second target sequence, and said number of said sequencing cycles of step b) is more (i.e., higher) than step e). In one embodiment, the number of said sequencing cycles of steps b) and e) is the same. In one embodiment, said hybridizing said one or more first member of said plurality of sequencing primers of step ii) a) is in the absence of said hybridizing said one or more second member of said plurality of sequencing primers of said step ii) d). For example, as shown in FIG. 8, a first member (e.g. Seq 1) and second member (e.g. Seq 2) of said plurality of sequencing primers are not hybridized at substantially the same time. In one embodiment, said hybridizing of two or more of said first member of said plurality of sequencing primers of step ii) a) is substantially at the same time (FIG. 8). In one embodiment, said hybridizing of two or more of said second member of said plurality of sequencing primers of step ii) d) is substantially at the same time (FIG. 8). In one embodiment, said rolony is generated from a DNA template comprising one or both of standard circle and dumbbell circle. In one embodiment, the method further comprises hybridizing, for each sequencing event, at least one of said plurality of sequencing primers to said member of said plurality of target sequences to start a sequencing read. For example, FIG. 9 shows one embodiment using typical library constructs for Illumina sequencer for pair-end sequencing. Based on the methods and strategies described herein and in provisional patent application No. 62/814,417 (Title: Compositions And Methods For Adaptor Design And Nucleic Acid Library Construction For Rolony-Based Sequencing), incorporated by reference, two rolonies (Rolony 1 and Rolony 2) can be generated based on the circle from the top-strand or the circle from the bottom strand in two separate tubes. The rolonies can be seeded or hybridized on the same flow cell or different areas of the same flow cell. For each sequencing event, two different sequencing primers are hybridized to the rolonies on the flow cell. After sequencing, the sequencing fragments are removed, and then another two different sequencing primers are hybridized to the rolonies on the flow cell. By this method, both strands can be sequenced to generate pair-end reading. The only requirements for library construct design are: there are no interactions or complementarity between each pair of sequencing primers that are applied and/or hybridized at the same time on the flow cells. In one embodiment, said sequentially sequencing said plurality of target sequences comprises one more of i) single-end sequencing on rolonies generated from a standard circle, said rolonies comprising multiple separate fragments to be sequenced, ii) pair-end sequencing on rolonies generated from a dumbbell circle, and iii) pair-end sequencing on rolonies generated from a standard circle of a library, said different strands comprising a top strand and a bottom strand.

The invention also provides a method for sequentially sequencing a single-stranded DNA sequence that contains at least two target sequences, said method comprising
i) providing
   a) a single-stranded DNA sequence comprising at least a first target sequence operably fused to a first oligonucleotide sequence, and a second target sequence operably fused to a second oligonucleotide sequence, and
   b) a first sequencing primer that is complementary to and specifically binds with said first oligonucleotide sequence, and a second sequencing primer that that is complementary to and specifically binds with said second oligonucleotide sequence,
ii) sequentially sequencing said first target sequence and said second target sequence, said sequentially sequencing comprises
   a) hybridizing said first sequencing primer to said first oligonucleotide sequence in a first reaction mixture to produce a first hybridized sequencing primer,
   b) extending said first hybridized sequencing primer to produce a first sequencing read, said extending comprises producing a first double-stranded DNA sequence containing a complement of said first target sequence operably fused to said first sequencing primer, and comprises sequencing at least one strand of said first double-stranded DNA sequence,
   c) removing the sequenced at least one strand of said first double-stranded DNA sequence produced in step b) from said first reaction mixture,
   d) hybridizing said second sequencing primer to its complementary said second oligonucleotide sequence in a second reaction mixture, to produce a second hybridized sequencing primer,
   e) extending said second hybridized sequencing primer to produce a second sequencing read, said extending comprises producing a second double-stranded DNA sequence containing a complement of said second target sequence operably fused to said second sequencing primer, and comprises sequencing at least one strand of said second double-stranded DNA sequence.
      In one embodiment, the method further comprises repeating steps c) to e) to sequence members of said target sequences that are different from both said first target sequence and said second target sequence. In one embodiment, said method lacks using a blocking reagent. In one embodiment, said method lacks addition of one or more said blocking reagent to both said first and second reaction mixtures. In one embodiment, said method lacks incorporation of one or more said blocking reagent into any of said first double-stranded DNA sequence produced in step b) and of said second double-stranded DNA sequence produced in step e). In one embodiment, said removing of step c) comprises washing with a buffer having a temperature higher than a melting temperature of said first double-stranded DNA sequence produced in step b). In one embodiment, said removing of step c) comprises washing with a buffer having low ionic strength. In one embodiment, said removing of step c) comprises washing with a buffer comprising proteins having 3′ to 5′ exonuclease activity, and/or enzyme having 5′ to 3′ exonuclease activity (irrespective of the phosphorylation status of polynucleotide produced in step b)), and/or compound that denatures double-stranded DNA. In one embodiment, said removing of step c) comprises washing with a buffer comprising a compound that denatures DNA, or reducing melting temperature of double-stranded DNA. In one embodiment, said double-stranded DNA sequence comprises at least a portion of a rolony. In one embodiment, said double-stranded DNA sequence comprises the extended sequencing segment. In one embodiment, said sequencing steps b) and e) comprises at least two sequencing cycles. In one embodiment, the number of said sequencing cycles of steps b) and e) is different. In one embodiment, said first target sequence is shorter (i.e., less or lower) than said second target sequence, and said number of said sequencing cycles of step b) is less (i.e., lower) than step e). In one embodiment, said first target sequence is longer than said second target sequence, and said number of said sequencing cycles of step b) is more (i.e., higher) than step e). In one embodiment, the number of said sequencing cycles of steps b) and e) is the same. In one embodiment, said hybridizing said first sequencing primer of step ii) a) is in the absence of said hybridizing said second sequencing primer of said step ii) d). For example, as shown in FIG. 8, a first member (e.g. Seq 1) and second member (e.g. Seq 2) of said plurality of sequencing primers are not hybridized at substantially the same time.

The invention further provides a kit for use in any one or more of the methods herein, such as for sequentially sequencing single-stranded DNA sequence that comprises a plurality of target sequences, wherein each member of said plurality of target sequences is operably fused to an oligonucleotide sequence, said kit comprising a) a plurality of sequencing primers, wherein each member of said plurality of sequencing primers is complementary to and specifically binds with a different segment of said oligonucleotide sequence, b) a reagent for removing sequencing fragments (such as denature reagent, degradation reagent, etc.), and c) instructions for using said plurality of sequencing primers and said reagent. In one embodiment, the kit further comprises one or more of d) a reagent for denaturing sequencing fragments (such as formamide, betaine, low ion strength wash buffer, etc.), e) a reagent for degradation of sequencing fragments (such as Exonuclease III, enzymes with 5′ to 3′ or 3′ to 5′ exonuclease activities, etc.), f) a reagent for removing sequencing primers that do not specifically bind with said different segment of said oligonucleotide sequence (such as low ion strength wash buffer), g) a reagent for removing said denaturing reagent (such as low ion strength wash buffer), and h) a reagent for removing said degradation reagent (such as low ion strength wash buffer).

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1. Sequential sequencing on different circle structures. There are two kinds of circle structure as a template for RCA, standard circle and dumbbell circle. As an example, two sequencing primers are designed for each circle structure. The corresponding rolony structure with two sequencing primers is shown for both cases of circle.

FIG. 2. Rolony concatemer schema for sequential sequencing. 2A. Double-stranded DNA template for circularization. There are 5 regions for each library fragment. #2 and #4: to be sequenced; #1 and #5: for bridge oligo ligation; #1, #3, #5 are conserved regions, good for amplification primer and sequencing primer design. 2B. Circled single-stranded DNA template for RCA with an example of RCA amplification primer. 2C. One concatemer unit of rolony. Black arrows represent sequencing primer, seq 1 and seq 2. 2D. Circled single-stranded DNA template for RCA with an example of bridge oligo.

FIG. 3. Flow chart of the invention's sequential sequencing process (exemplified with two sequencing primers).

FIG. 4. Phi29 DNA polymerase 3′ to 5′ exonuclease activity. 4A. Oligo design schema for capillary electrophoresis (CE) fragment analysis. Green star represents the position of FAM (fluorescein amidite) internal labeled nucleotide. 4B. Quantitative results of CE fragment analysis. 4C. Electropherogram results of CE fragment analysis.

FIG. 5. Removing fragment by low ionic strength wash buffer. A tile image file from flow cell at cycle 1 versus cycle 6. The image is from ImageJ software.

FIG. 6. Results of second sequencing of sequential sequencing (sample index sequencing).

FIG. 7. Evaluation Matrix for sequential sequencing. FIG. 7A. Data analysis process without demultiplex (Analysis Path 1) and with demultiplex (Analysis Path 2). FIG. 7B. The relationship of raw reads. T=M+N=E+F=A+B+C+D. T: total reads after duplicates removal M: mapped reads, output of Analysis Path 1 after mapping. N: unmapped reads E: reads with sample index, output of Analysis Path 2 after demultiplex. F: reads without sample index, output of Analysis Path 2 after demultiplex. A: mapped reads with sample index, output of Analysis Path 2 after demultiplex and mapping B: mapped reads without sample index C: unmapped reads with sample index D: unmapped reads without sample index.

FIG. 8. Using multiple different sequencing primers to hybridize to a target sequence, exemplified by a rolony sequence. Rolony 1 and 2 can be from different sources, such as one from human genome, another from E. coli, and/or one from human BRCA target, another from human Lung target, and/or one from top-strand, another from bottom-strand for pair-end sequencing. Sequencing primers Seq 1 and Seq 2 hybridize with rolony 1, and sequencing primers Seq 3 and Seq 4 hybridize with rolony 2. There are no interactions between Seq 1 and Seq 3 (for the first segment sequencing). There are no interactions between Seq 2 and Seq 4 (for the second segment sequencing).

FIG. 9. Pair-end sequencing with sequential sequencing. It shows a typical library construct comprising multiple distinct segments (umi, sample index, insert, etc.). Two kinds of rolonies can be generated from either top-strand or bottom-strand of the double-stranded DNA library construct with only one-strand specifically pre-phosphorylated for ligation to form circle. Two kinds of rolonies can be co-seeded on the same flow cell to perform sequential sequencing, with an example order listed in the table. For each sequencing process, multiple sequencing primers can be hybridized on the same flow cell to sequencing different target sequencings of different kinds of rolonies, shown in the table.

FIG. 10 shows schematically show one embodiment of a flow cell. FIG. 10A shows a three dimensional translucent view of a flow cell, comprising fluid tubing connections, cartridge heaters, and 0-ring seal. FIG. 10B is a two dimensional drawing of a side view of a flow cell, showing an array or slide with spaced spots on the surface (representing positions for biomolecules and/or anchoring molecules), said array positioned in a fluid channel such that solutions of buffers and/or reagents can be introduced over the surface under conditions whereby reactions and/or washing can be achieved. The arrows show one preferred direction of fluid flow, with entrance and exit ports, as well as one preferred method of sealing (0-ring seal).

### DEFINITIONS

To facilitate understanding of the invention, a number of terms are defined below.

In some embodiments, as used herein, “sequential sequencing” generally refers to a sequencing process that uses and/or requires multiple (i.e., two or more) different sequencing primers in a sequencing event on the same flow cell. After binding of each sequencing primer to the template, a sequencing process start, which includes sequencing/reading multiple nucleotides (in the case of SBS, it involves multiple cycles of synthesis/extend, image, cleave and wash). There are multiple sequencing processes for one flow cell in a continuous workflow.

In some embodiments, as used herein, “single-stranded DNA sequence” generally refers to a “sense strand” (i.e., “coding strand”) or an “antisense strand” (i.e., “template strand”) of a double-stranded DNA. The sense strand runs from 5′ to 3′, and the antisense strand runs from 3′ to 5′.

In some embodiments, as used herein, “complement” and “complementary” when in reference to a sequence of interest may be used interchangeably, and generally refer to a nucleic acid sequence that can form a double-stranded structure with the sequence of interest by matching base pairs. For example, the complementary sequence to 5′-G-T-A-C-3′ is 3′-C-A-T-G-5′. In one embodiment, PCR primers are 100% complementary along their entire length to a region of a target polynucleotide.

In some embodiments, as used herein, “amplification” generally refers to making copies of polynucleotide sequences of interest. Amplification methods include both thermocycling (such as “polymerase chain reaction” (“PCR”)) amplification) and isothermal amplification that does not require thermocycling (such as described in application number WO07107710), using a commercially available Solexa/Illumina cluster station as described in PCT/US/2007/014649. The cluster station is essentially a hotplate and a fluidics system for controlled delivery of reagents to a flowcell. Cluster station is not required for rolony based clonal amplification.

In some embodiments, as used herein, a “clonal amplification,” “clonally amplified,” and grammatical equivalents when in reference to a nucleotide sequence generally refer to generation of multiple copies of the nucleotide sequence.

In some embodiments, as used herein, “amplicon” generally refers to a nucleotide sequence that is the source and/or product of amplification or replication events. It can be formed artificially, using various methods including polymerase chain reactions (PCR) or ligase chain reactions (LCR), or naturally through gene duplication.

In some embodiments, as used herein, “polymerase chain reaction” (“PCR”) generally refers to a method for making copies of a specific DNA segment using repeated thermal PCR cycles. “PCR cycle” refers to a combination of denaturing a double-stranded template DNA by heating to separate it into two single strands, annealing the DNA primers to the template DNA by lowering the temperature, and extending the new DNA strand by a polymerase enzyme and by raising the temperature.

In some embodiments, as used herein, “hybridizing” and grammatical equivalents generally refer to a process by which single-stranded DNA or RNA molecules anneal to complementary single-stranded DNA or RNA through base pairing. Hybridization and the strength of hybridization (i.e., the strength of the association between the nucleic acids) are impacted by such factors as the degree of complementarity between the nucleic acids, stringency of the conditions involved, the melting temperature (Tₘ) of the formed hybrid, and the G:C ratio within the nucleotide sequence. Conditions for hybridizing DNA molecules, such as primers and target DNA polynucleotides are known in the art.

In some embodiments, as used herein, “operable,” “operably, “operable combination” and “operably linked,” when in reference to the relationship between nucleic acid sequences may be used interchangeably, and generally refer to fusing the sequences in frame such that they perform their intended function. For example, operably linking a promoter sequence to a nucleotide sequence of interest refers to fusing the promoter sequence and the nucleotide sequence of interest in a manner such that the promoter sequence is capable of directing the transcription of the nucleotide sequence of interest and/or the synthesis of mRNA encoded by the nucleotide sequence of interest, and also such that the nucleotide sequence of interest retains its function, such as of encoding mRNA.

In some embodiments, as used herein, “fuse,” “fusion,” and grammatical equivalents when made in reference to a first and second nucleotide sequences may be used interchangeably, and generally refer to the linkage of the first and second nucleotide sequences via phosphodiester bonds. Fusion of a first and second nucleotide sequences may be direct or indirect. “Direct” fusion refers to the absence of intervening nucleotides between the first and second nucleotide sequences. “Indirect” fusion refers to the presence of one or more nucleotides between the first and second nucleotide sequences. For example, the term first sequence “fused at its 3′ end” to a second sequence refers to a first sequence that is fused, directly or indirectly, at its 3′ end to the second sequence.

In some embodiments, as used herein, “plurality” generally refers to a population of two or more different polynucleotides or other referenced molecule. Accordingly, unless expressly stated otherwise, the term “plurality” is used synonymously with “population.” A plurality includes 2, 3, 4, 5, 6, 7, 8, 9, 10, 20, 30, 40, 50, 60, 70, 80, 90 or a 100 or more different members of the population. A plurality also can include 200, 300, 400, 500, 1000, 5000, 10000, 50000, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶ and/or 1×10⁷, or more different members. A plurality includes all integer numbers in between the above exemplary population numbers.

In some embodiments, as used herein, “member” of a plurality of sequences generally refers to sequences having the same sequence, i.e., the same arrangement of nucleotides. This is exemplified by sequences having the same adapter sequence, same index sequence, same barcode sequence, same universal sequence, same genomic sequence, same sequencing primer sequence, etc.)

In some embodiments, as used herein, “target sequence” generally refer to a polynucleotide sequence that is the object of an analysis or action. “Target sequence” includes members of a plurality of target sequences having the same sequence. The analysis or action includes subjecting the polynucleotide to copying, amplification, sequencing and/or other procedure for nucleic acid interrogation. In some embodiments, a target sequence comprises a first target sequence having a known or predetermined nucleotide sequence (such as an adapter sequence, index sequence, barcode sequence, universal sequence, etc.) that is adjacent to a second target sequence having an unknown sequence that is to be determined (which may be referred to as an “unknown sequence”), such as a portion of a genomic sequence. A target sequence can be of any appropriate length. In some embodiments, a target sequence is double-stranded or single-stranded. In some embodiments, a target sequence is DNA or RNA (e.g., mRNA, rRNA, tRNA, cfDNA, cfRNA, long non-coding RNA, microRNA).

In some embodiments, as used herein, “insert sequence” and “regions of interest” may be used interchangeably, and generally refer to a polynucleotide sequence that is the object to be sequenced, but not including the sequences for sample index, barcodes, or conserved sequences for sequencing primer hybridization.

In some embodiments, as used herein, “adapter,” “adaptor,” and “linker” may be used interchangeably, and generally refer to a short, chemically synthesized, single-stranded or double-stranded oligonucleotide that can be ligated to the 3′ and/or 5′ ends of other DNA or RNA molecules. Adapters containing specific sequences designed to interact with next-generation-sequencing (NGC) platforms (such as the surface of the flow-cell or beads may be ligated to one or both of the 3′ and 5′ ends of target polynucleotides prior to sequencing. For example, adapters include “indexed adapters” and “universal adapters.” The primary function of both indexed adapters and universal adapters is to allow any DNA sequence to bind to a flowcell for next generation sequencing (NGS), and to allow for PCR enrichment of only adapter ligated DNA sequences for cluster generation (such as either on a MiSeq flowcell or on an Ion Torrent bead). The addition of indexes unique to each sample allows for the mixing of two or more samples, for sequencing to occur, and for results to be analyzed after the sequencing is complete. The structure of adapters is dictated by the sequencing platform. In some embodiments, as used herein, “Indexed adapters” (also referred to as “index adapters”) contain index polynucleotide sequences, and are known in the art as exemplified by TruSeq Indexed Adapter: 5′ P*GATCGGAAGAGCACACGTCTGAACTCCAGTCAC ATCTCGTATGCC 3′. Indexed adapters allow for indexing or “barcoding” of samples so multiple DNA libraries can be mixed together into one sequencing lane (known as multiplexing). Methods for designing and making index adapters are known in the art (Illumina TruSeq Adapters Demystified Rev. A, © 2011 Tufts University Core Facility). In some embodiments, as used herein, “universal adapters” contain universal polynucleotide sequences, and are known in the art as exemplified by TruSeq Universal Adapter: 5′AATGATACGGCGACCACCGAGATCTACACTCTTTCCCTACACGACGCTCTTCCGATC*T 3′. The stars (*) in the above TruSeq Indexed Adapter and TruSeq Universal Adapter indicate a phosphorothioate bond between the last C and T to prevent cleaving off the last T that is needed for annealing the overhang. The phosphate group on the indexed adapter is required to ligate the adapter to the DNA fragment. The in the above exemplary TruSeq Indexed Adapter represents the “sample index.” The last 12 bases are complementary if the Indexed Adapter is reversed. Methods for designing and making universal adapters are known in the art (Illumina TruSeq Adapters Demystified Rev. A, © 2011 Tufts University Core Facility).

In some embodiments, as used herein, “index,” “index sequence,” and “sample index” may be used interchangeably, and generally refer to a region of an adapter nucleic acid sequence and that is useful as an identifier for the population to which the ligated nucleic acid sequence belongs. In some embodiments, an index comprises a fixed nucleic acid sequence that may be used to identify a collection of sequences belonging to a common library. An index sequence has a unique nucleotide sequence that is distinguishable from the sequence of other indices as well as the sequence of other nucleotide sequences within polynucleotides contained within a sample. An index sequence is useful where different members of the same molecular species can contain the same index sequence and where different species within a population of different polynucleotides can have different unique indices. An index sequence can be a random or a specifically designed nucleotide sequence. An index sequence can be of any desired sequence length so long as it is of sufficient length to be a unique nucleotide sequence within a plurality of indices in a population and/or within a plurality of polynucleotides that are being analyzed or interrogated. A nucleotide index is useful, for example, to be attached to a target polynucleotide to tag or mark a particular species for identifying all members of the tagged species within a population. In some embodiments, index sequences enable sequencing of multiple different samples in a single reaction (e.g., performed in a single flow cell). In some embodiments, an index sequence can be used to orientate a sequence imager for purposes of detecting individual sequencing reactions. In some embodiments, an index sequence may be 2 to 25 nucleotides in length. Methods for designing and making index sequences are known in the art (Illumina TruSeq Adapters Demystified Rev. A, ° 2011 Tufts University Core Facility), and U.S. Pat. No. 9,926,598.

In some embodiments, as used herein, “barcode,” “barcode sequence,” “molecular barcode,” and“umi” may be used interchangeably, and generally refer to a region of an adapter nucleic acid sequence that is useful as a unique identifier for the specific nucleic acid to which it is ligated. In some embodiments, a molecular barcode comprises a randomized nucleic acid sequence that provides a unique identifier for the nucleic acid to which it is ligated. In some embodiments, a molecular barcode may be used to identify unique fragments and “de-duplicate” the sequencing reads from a sample. In some embodiments, a molecular barcode may be used to identify and remove PCR or isothermal amplification duplicates. In some embodiments, a molecular barcode may be 2 to 25 nucleotides in length.

In some embodiments, as used herein, “universal sequence” and “universal polynucleotide sequence” may be used interchangeably, and generally refer to a sequence that enables amplification of any target polynucleotide of known or unknown sequence that has been modified to enable amplification with the universal primers. In one embodiment, such amplification produces an amplified target polynucleotide containing a “universal” sequence, such as a universal adapter sequence, at the target polynucleotides' 3′ and/or 5′ ends. The attachment of universal known ends to a library of DNA fragments by ligation allows the amplification of a large variety of different sequences in a single amplification reaction. The sequences of the known sequence portion of the nucleic acid template can be designed such that type 2s restriction enzymes bind to the known region, and cut into the unknown region of the amplified template. Universal primers are known in the art and exemplified by Illumina's Sequences S1 and S2 which, in combination, direct amplification of a template by solid-phase bridging amplification reaction. The template to be amplified must itself comprise (when viewed as a single strand) at the 3′ end a sequence capable of hybridizing to sequence S1 in the forward primers and at the 5′ end a sequence the complement of which is capable of hybridizing to sequence S2 the reverse primer. Methods for designing and making universal sequences are known in the art (Illumina TruSeq Adapters Demystified Rev. A, © 2011 Tufts University Core Facility), and U.S. Pat. No. 8,765,381.

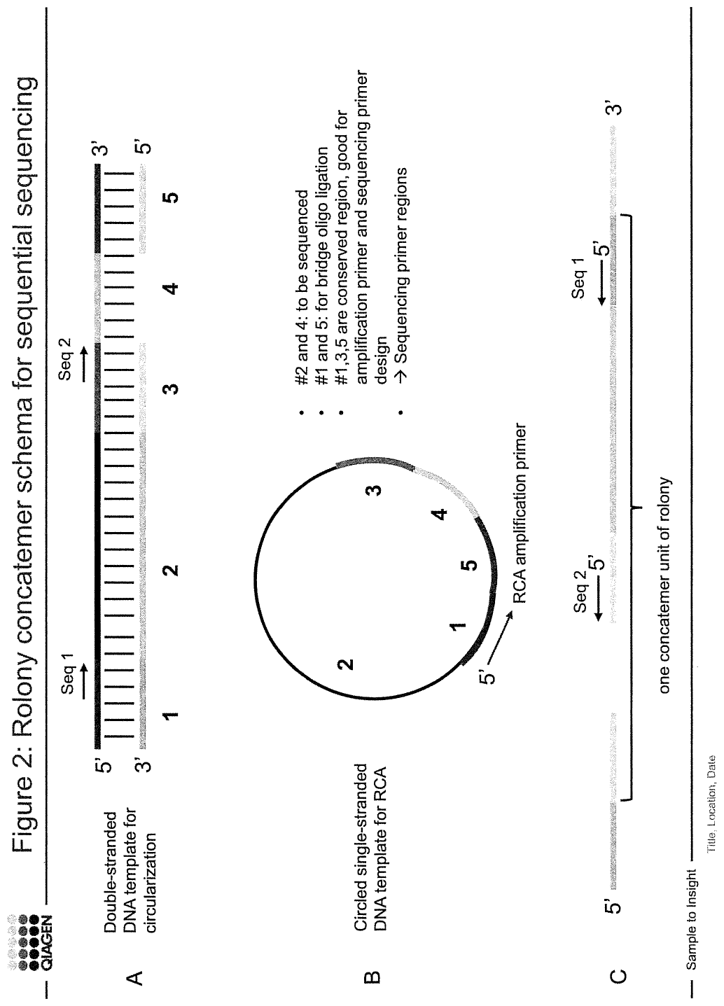

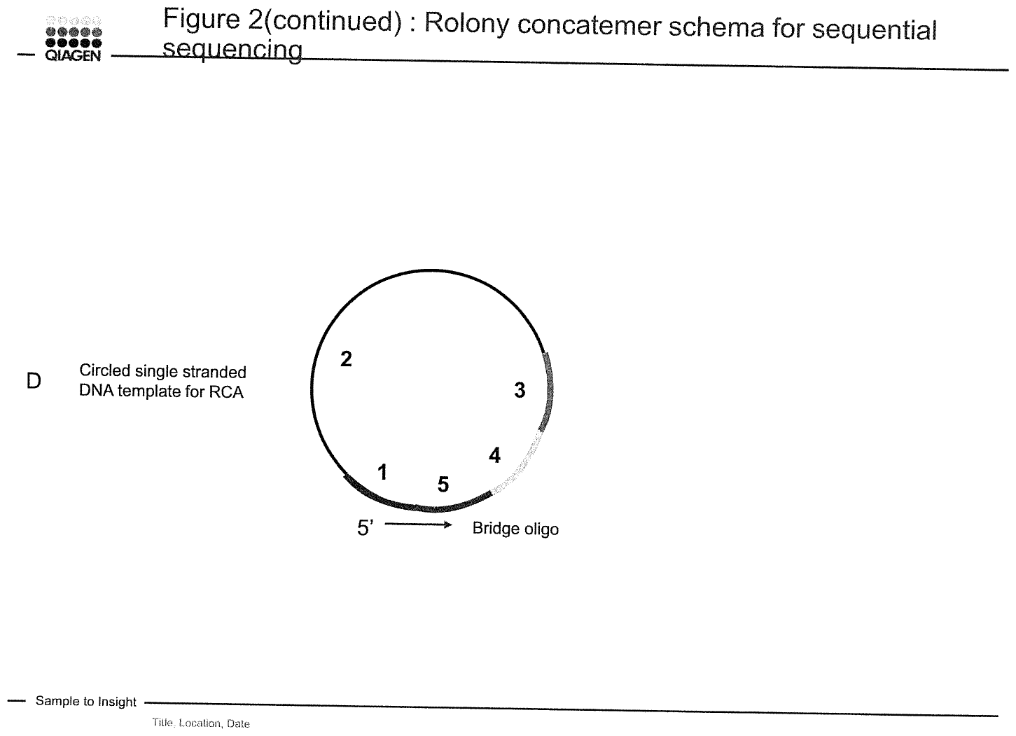

In some embodiments, as used herein, “oligonucleotide sequence” generally refers to a molecule containing more than two (2) deoxyribonucleotides or ribonucleotides, including for example from two (2) to one hundred (100), preferably from ten (10) to fifty (50), and more preferably from twenty (20) to thirty (30) deoxyribonucleotides or ribonucleotides. In one embodiment, an oligonucleotide sequence is complementary to, and specifically hybridizes with, a sequencing primer. Oligonucleotide sequences are exemplified in FIG. 2 regions 1, 3, and 5.

In some embodiments, as used herein, a “primer” sequence generally refers to a short single-stranded DNA that hybridizes to a target polynucleotide sequence, and serves as a starting point for synthesis of a complementary strand of the target polynucleotide sequence. “PCR primer” is a primer used in a “polymerase chain reaction” (“PCR”). Design principles for PCR primers are known in the art, including primer length, specificity to the target polynucleotide sequence, melting temperature (Tₘ) value, annealing temperature (Tₐ), freedom of strong secondary structures and self-complementarity, and G:C content.

In some embodiments, as used herein, “target specific” and “site specific” when used in reference to a primer or other oligonucleotide sequence is intended to mean a primer or other oligonucleotide sequence that includes a nucleotide sequence that is complementary to, and that specifically and selectively hybridizes (i.e., anneals) to, at least a portion of a target polynucleotide sequence. Target specific primers include forward and reverse primers, universal primers, index primers, sequencing primers, and the like.

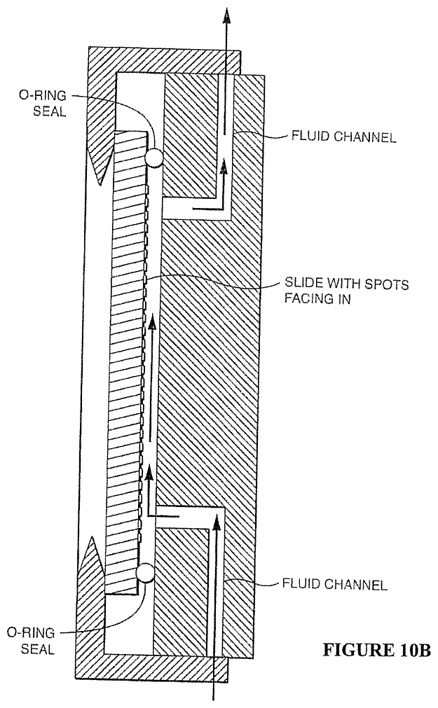

In some embodiments, as used herein, a “flow cell” is a vessel where the sequencing chemistry occurs. In some embodiments, the flow cell is a glass slide containing small fluidic channels, through which polymerases, dNTPs and buffers can be pumped. In some embodiments, the glass inside the channels is decorated with short oligonucleotides complementary to the adapter sequences. In some embodiments, the DNA library containing adapters is diluted and hybridized to these oligonucleotides, temporarily immobilizing individual DNA strands onto the flow cell. In some embodiments, library strands are amplified using a “bridge-PCR” strategy employing cycles of primer extension followed by chemical denaturation. Through the in-situ amplification process, the strands are amplified by several thousand. In some embodiments, DNA libraries are hybridized to the flow cell in low molar quantities (6-20 pM). This results in a large physical separation between template DNA strands. At the end of amplification, small clusters of identical DNA are left as molecules immobilized on a 2D surface, which can be sequenced en masse. In some embodiments, sequencing may proceed by repeating the following cycle of steps: First, a single base containing a fluorophore and 3′ blocking moiety is incorporated by a polymerase. Then, the flow cell is imaged using fluorescent microscopy. Then, the fluorescent and blocking moieties are cleaved, allowing the next base to be incorporated. For rolony based sequencing, there is no need for grafted oligos on the surface of flow cell. Rolony can bind directly on flow cell based on electro charge. An exemplary flow cell is shown in FIG. 10, and U.S. Publication Ser. No. US 2016-0369337.

In some embodiments, as used herein, “sequencing primer” generally refers to a primer sequence that is complementary to, and that specifically and selectively hybridizes (i.e., anneals) to, a portion of a template target sequence, and that is extended by incorporation of nucleotides to produce a double-stranded sequence that is complementary to the template target sequence, whereby the extended sequence is used to ascertain the order of nucleotides in the complementary template target sequence.

In some embodiments, as used herein, “sequencing cycle” in reference to Sequencing By Synthesis (SBS) refers to extending a primer sequence, imaging, cleaving, and washing. In some embodiments, as used herein, an SBS “sequencing cycle” refers to adding one nucleotide to a template DNA sequence. Optionally, the sequencing cycle further includes interrogating the nucleotides that are incorporated into the extended sequence to ascertain the order of the incorporated nucleotides in the complementary template target sequence.

In some embodiments, as used herein, “paired-end sequencing” and “pair-end sequencing” generally refer to sequencing both ends of a DNA fragment. This is in contrast to “single-read sequencing” that sequences one end of a DNA fragment. In some embodiments, paired-end sequencing requires the same amount of DNA as single-read sequencing, and produces twice the number of reads as single-read sequencing for the same time and effort in library preparation. Methods for paired-end sequencing and single-read sequencing are known in the art, such as those used by Illumina, Inc.

In some embodiments, as used herein, “blocking reagent” and “terminator reagent” may be used interchangeably, and generally refer to a dideoxynucleotide triphosphate (“ddNTP” also referred to as “blocking nucleotide”) lacking the 3′-OH group of a deoxynucleotide triphosphate (dNTP) that is essential for polymerase-mediated strand elongation in a polymerase chain reaction (PCR). Thus, ddNTPs are used in combination with a DNA polymerase (e.g., JBS Sequencing polymerase, Thermosequenase™, terminal transferase (New England Biolabs, USA)) as 3′-end chain terminators. The ddNTPs may be produced by reversibly or irreversibly capping the 3′-OH of a nucleotide with a moiety so that the 3′-O-modified nucleotide continues to be recognized by the DNA polymerase as substrate and may be incorporated into the synthesized DNA sequence. However, unlike the unmodified dNTP, DNA polymerase does not extend the 3′-O-modified ddNTP. Blocking reagents are known in the art, such as those used in sequencing by synthesis (SBS).

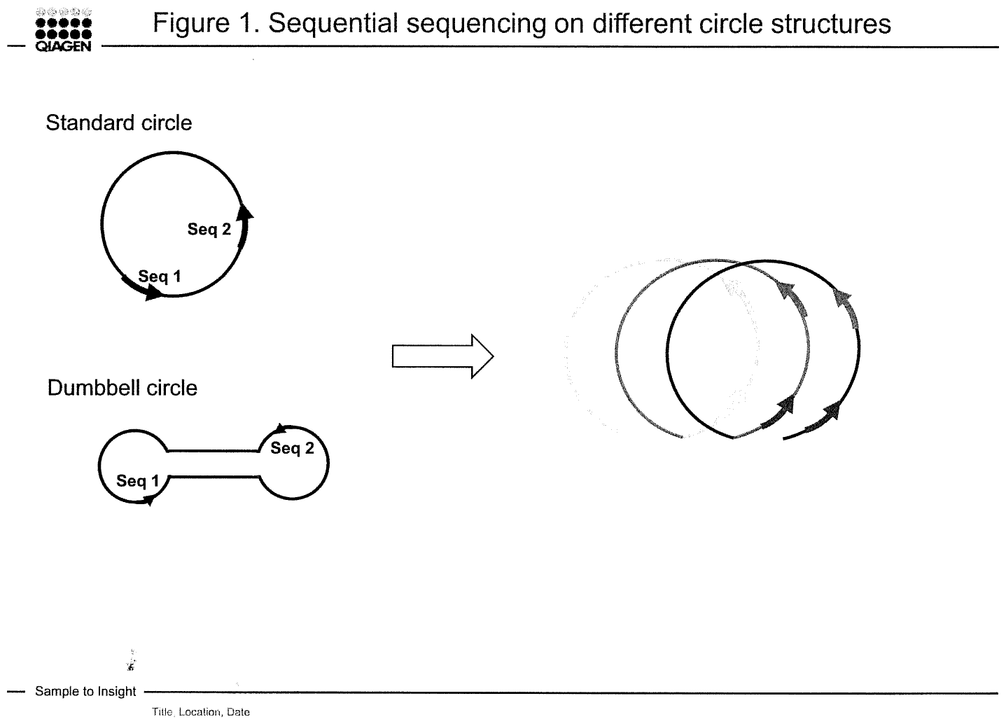

In some embodiments, as used herein, “rolony” and “nanoball” may be used interchangeably, and generally refer to DNA sequences generated by clonal amplification of a circularized DNA fragment to produce a single-stranded DNA with multiple copies of concatemers. The circularized DNA fragment includes a standard circle and dumbbell circle (FIG. 1). Methods for generating rolonies are known in the art, including the process of library construction, circularization, and amplification by RCA isothermal process. Rolonies may be sequenced using methods known in the art such as sequencing-by-synthesis (SBS) and/or sequencing-by-ligation (SBL, International Patent Application Publication No. WO2011/044437). RCA based clonal amplification can eliminate the need for emulsion PCR (ePCR) and thereby provide the option of eliminating an often expensive and labor-intensive step in many next generation sequencing methods.

In some embodiments, as used herein, “circular,” “circularized”, and grammatical equivalents when in reference to DNA generally refer to DNA that forms a closed loop and has no ends. Examples include naturally occurring and recombinant plasmids, covalently closed circular DNA (cccDNA) formed by some viruses inside cell nuclei, circular bacterial chromosomes, mitochondrial DNA (mtDNA), and chloroplast DNA (cpDNA).

In some embodiments, as used herein, “dumbbell circle” generally refers to a circle with double-stranded insert sequences between two looped-structures at both ends (FIG. 1).

In some embodiments, as used herein, “standard circle” generally refers to a circle other than the dumbbell circle (FIG. 1).

In some embodiments, as used herein, a “concatemers” is a continuous DNA molecule that contains multiple copies of the same DNA sequence linked in series. These polymeric molecules are exemplified by copies of an entire genome linked end to end and separated by cos sites (a protein binding nucleotide sequence that occurs once in each copy of the genome).

In some embodiments, as used herein, “bridge oligo” and “guide oligo” may be used interchangeably, and generally refer to DNA sequences (FIG. 2D) designed for circularization of single-stranded DNA. The bridge oligo includes the 5′-end and 3′-end complementary sequences of single-stranded DNA to be circled. The 5′-end and 3′-end of the single-stranded DNA hybridize, i.e., anneal to the bridge oligo. Therefore, the 5′-end and 3′-end are close to each other and can be ligated by ligase. In some embodiments, the 5′-end of single-stranded DNA is preferably phosphorylated before ligation to ensure ligation efficiency.

“Sequence by synthesis,” “sequencing-by-synthesis” and “SBS” may be used interchangeably, and generally refer to a DNA sequencing method that uses four fluorescently labeled nucleotides to sequence the tens of millions of clusters on the solid surface (flow cell, or a chip) in parallel. During each sequencing cycle, a single labeled modified deoxynucleoside triphosphate (dNTP) is added to the nucleic acid chain. The nucleotide label serves as a terminator for polymerization, so only a single base can be added by a polymerase enzyme to each growing DNA copy strand. After each dNTP incorporation, the fluorescent dye is imaged to identify the base and then chemically or enzymatically cleaved to allow incorporation of the next nucleotide. This chemistry is called “reversible terminators”. Since all four reversible terminator-bound dNTPs (A, C, T, G) are present as single, separate molecules, natural competition minimizes incorporation bias. For each SBS cycle, it includes at least extend, image and cleave steps.

“Sequence by ligation,” “sequencing-by-ligation” and “SBL” may be used interchangeably, and generally refer to a DNA sequencing method that uses the enzyme DNA ligase to identify the nucleotide present at a given position in a DNA sequence. Unlike most currently popular DNA sequencing methods, this method does not use a DNA polymerase to create a second strand. Instead, the mismatch sensitivity of a DNA ligase enzyme is used to determine the underlying sequence of the target DNA molecule. Sequencing by ligation relies upon the sensitivity of DNA ligase for base-pairing mismatches. The target molecule to be sequenced is a single strand of unknown DNA sequence, flanked on at least one end by a known sequence. A short “anchor” strand is brought in to bind the known sequence. A mixed pool of probe oligonucleotides is then brought in (eight or nine bases long), labeled (typically with fluorescent dyes) according to the position that will be sequenced. These molecules hybridize to the target DNA sequence, next to the anchor sequence, and DNA ligase preferentially joins the molecule to the anchor when its bases match the unknown DNA sequence. Based on the fluorescence produced by the molecule, one can infer the identity of the nucleotide at this position in the unknown sequence. The oligonucleotide probes may also be constructed with cleavable linkages which can be cleaved after identifying the label. This will both remove the label and regenerate a 5′ phosphate on the end of the ligated probe, preparing the system for another round of ligation. This cycle can be repeated several times to read longer sequences. This sequences every Nth base, where N is the length of the probe left behind after cleavage. To sequence the skipped positions, the anchor and ligated oligonucleotides may be stripped off the target DNA sequence, and another round of sequencing by ligation started with an anchor one or more bases shorter. In a simpler embodiment, albeit more limited, technique is to do repeated rounds of a single ligation where the label corresponds to different position in the probe, followed by stripping the anchor and ligated probe. Sequencing by ligation can proceed in either direction (either 5′-3′ or 3′-5′) depending on which end of the probe oligonucleotides are blocked by the label. The 3′-5′ direction is more efficient for doing multiple cycles of ligation, and is the opposite direction to polymerase based sequencing methods. SBL is exemplified by International Patent Application Publication No. WO2011/044437.

“Sequencing at least one strand” when in reference to a double-stranded DNA sequence in the invention's methods refers to determining the sequence of nucleotides in the at least one strand, such as by taking a picture of the chip to which the at least one strand is attached in order to distinguish the nucleotides by the color of the tags followed by using a computer to determine what base was added by the wavelength of the fluorescent tag, and recording it for every spot on the chip.

In some embodiments, as used herein, “sequencing segment” and “sequencing fragment” may be used interchangeably, and generally refer to the nucleotide sequences formed by a serial of sequencing cycles (SBS, or SBL, etc.) from the sequencing primer hybridization sites. Generally, a sequencing segment contains at least 2 nucleotides and up to hundreds of nucleotides.

In some embodiments, as used herein, buffer having “low ionic strength” generally refers to a buffer having less than, or equal to, 50 mM of each of sodium chloride and Mg++, including a zero amount of sodium chloride and Mg++.

In some embodiments, as used herein, “Tₘ” and “melting temperature” may be used interchangeably, and generally refer to the temperature at which the oligonucleotide is 50% annealed to its complementary sequence. Tₘ depends on the length of the DNA molecule, its specific nucleotide sequence and buffer components, etc. Algorithms to estimate Tₘ are known in the art, such as those from Integrated DNA Technologies, USA.

“Efficiency” when in reference to an amplicon refers to the percentage of total reads of the amplicon. “Higher efficiency” refers to an increase in the percentage of total reads, exemplified by an increase of at least 0.1 fold (i.e., 10%), including an increase of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 fold, and exemplified by an increase from 0.1 fold (i.e., 10%) fold to 100 fold (i.e., 10,000 fold), including from 0.1 to 90 fold, 1 to 90 fold, 1 to 80 fold, 1 to 70 fold, 1 to 60 fold, 1 to 50 fold, 1 to 40 fold, 1 to 30 fold, 1 to 29 fold, 1 to 28 fold, 1 to 27 fold, 1 to 26 fold, 1 to 25 fold, 1 to 24 fold, 1 to 23 fold, 1 to 22 fold, 1 to 21 fold, 1 to 20 fold, 1 to 19 fold, 1 to 18 fold, 1 to 17 fold, 1 to 16 fold, 1 to 15 fold, 1 to 14 fold, 1 to 13 fold, 1 to 12 fold, 1 to 11 fold, 1 to 10 fold, 1 to 9 fold, 1 to 8 fold, 1 to 7 fold, 1 to 6 fold, 1 to 5 fold, 1 to 4 fold, 1 to 3 fold, and 1 to 2 fold.

In some embodiments, as used herein, the terms “higher,” “greater,” and grammatical equivalents (including “increase,” “elevate,” “raise,” etc.) when in reference to the level of any molecule (e.g., nucleotide sequence, amplicon, nucleic acid sequence, amino acid sequence, etc.) and/or phenomenon (e.g., efficiency, amplification of a nucleotide sequence, expression of a gene, etc.), specificity of binding of two molecules (e.g., binding of a director to a driver), in a first sample relative to a second sample, may be used interchangeably, and generally mean that the quantity of the molecule and/or phenomenon in the first sample is higher than in the second sample by any amount that is statistically significant using any art-accepted statistical method of analysis.

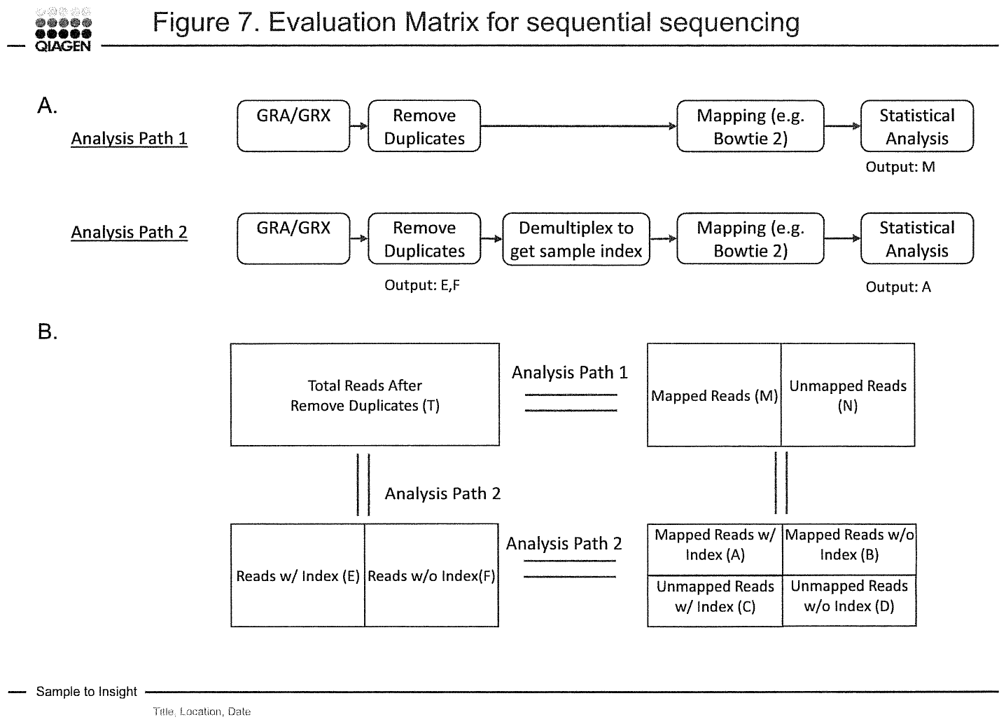

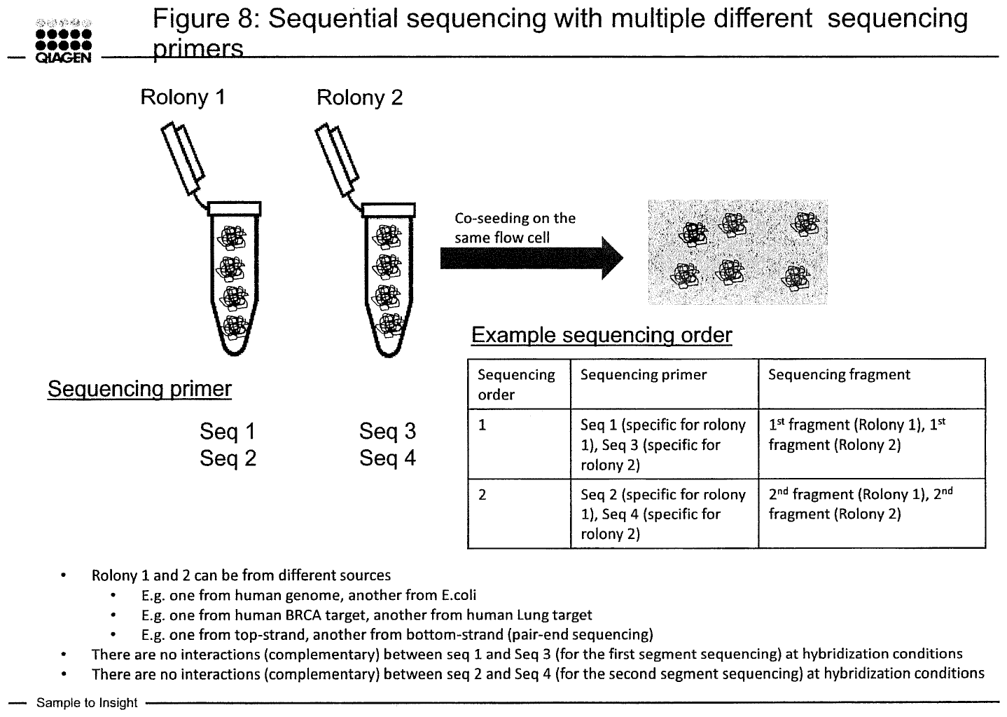

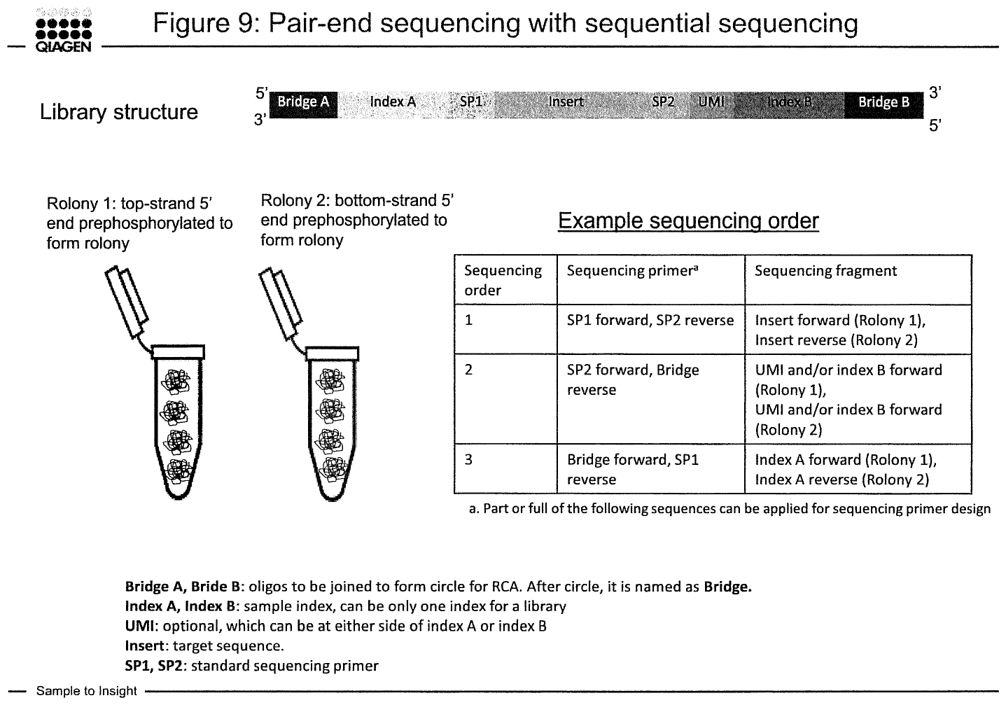

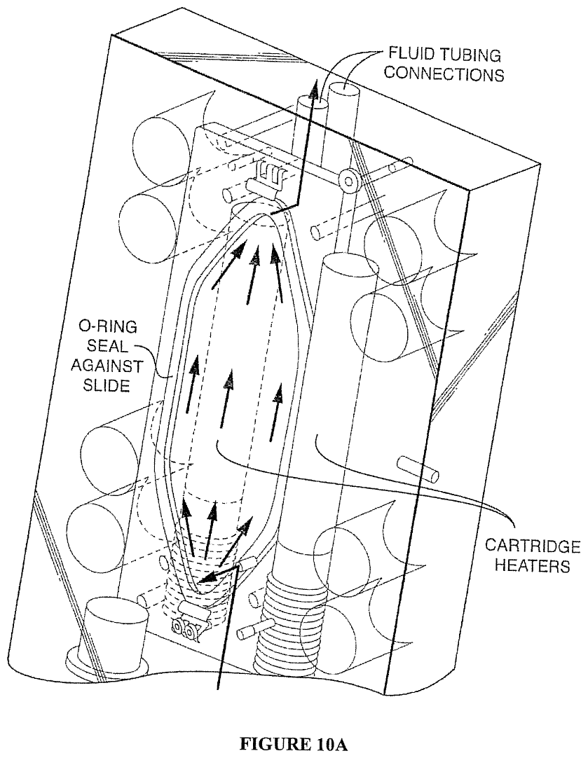

In some embodiments, as used herein, “percent of index” and “% index” may be used interchangeably, and generally refers to the percentage of indexed raw reads in the number of total raw reads (without quality filtering and mapping). It represents the success of second segment sequencing. The higher, the better. The minimal acceptance criterion is 50%. % barcodes=E/(E+F), shown in FIG. 7.

In some embodiments, as used herein, “percent of aligned” and “% aligned” may be used interchangeably, and generally refers to the percentage of total mapped reads after demultiplex over total mapped reads without demultiplex. It represents the success of second segment sequencing. The higher, the better. The minimal acceptance criterion is 75%. % aligned=A/M, shown in FIG. 7.

In some embodiments, as used herein, “Q-score ratio” generally refers to the ratio of the average Q-score of cycle 3 to 8 (inclusive) of the 1st sequencing segment over the average Q-score of cycle 2 to 7 (inclusive) of the 2nd sequencing segment. It represents the success of second segment sequencing. The higher, the better. The minimal acceptance criterion is 75%.

### DESCRIPTION OF THE INVENTION

The invention provides methods that solve prior art problems with regard to sequential sequencing of a polynucleotide template, exemplified by, but not limited to, sequencing on rolony. Comparing to the prior art approaches, such as blocking the previous sequencing fragment by nucleotide blocking with enzymes and ddNTP, the invention's methods have the advantages of low cost, shorter turn-around-time, high efficiency, and easy implementation. For example, the cost of reagents for the invention's methods is approximately only 1.5% to 2.0% of the cost of reagents for a prior method that uses a combination of enzymes and ddNTP to block the previous sequencing fragment.

The invention provides, in one embodiment, a method for sequential sequencing of a polynucleotide template, exemplified by, but not limited to, sequencing on rolony, for binding of sequencing primer. The rolony can be sequenced with multiple different sequencing primers for different target sequences, such as: insert sequences from a sample (regions of interest), index sequences, barcode sequences (or umi, unique molecular identifiers), universal sequences, etc. This sequential sequencing workflow is independent of the process of sequencing blocking which is cost expensive and relies on enzymes and blocking nucleotides (e.g. ddNTP). The invention's methods comprise sequentially binding sequencing primer to a polynucleotide template that contains fragments to be sequenced, sequencing each fragment (such as insert, or sample index, or barcode, etc.) and removing (such as by washing-off/denaturing with a wash buffer) the previously sequenced (i.e., extended) fragments.

One of the unique features of using rolony-based sequencing in the instant invention includes clonal amplification first (preferably in solution, not on flow cell surface) followed by hybridizing the rolony on a flow cell surface. In one preferred embodiment, only the sequencing step is on a flow cell surface. It distinguishes the invention's methods from bridge-amplification based technology, with which clonal amplification and sequencing are both on flow cell surface. Therefore, in a preferred embodiment, there are no grafted oligos on a flow cell surface for the invention's rolony-based sequencing.

Thus, in one embodiment, the invention provides a method for sequential sequencing of a polynucleotide template, such as a ssDNA rolony (DNA nanoball) that contains a concatemer created by rolling circle amplification (RCA) with single or multiple sequencing primer hybridization sites. Each unit of concatemer may contain a sample index, barcodes (umi) and a targeted insert sequences (regions of interests). The sequencing technology is based on SBS (sequence-by-synthesis) or SBL (sequence-by-ligation). The template for sequencing should be single-stranded DNA. Currently, there are generally two ways to make the single-stranded DNA: a) Rolony (shown in the examples); b) bridge-amplification and emulsion PCR to generate double-stranded DNA first, and sodium hydroxide denaturation to generate single-stranded DNA.

Generally, in one embodiment, the invention's methods include 1) hybridizing the first sequencing primer for segment 1; 2) sequencing the segment 1; 3) removing the first sequencing fragments by wash buffer at a certain temperature in the sequencing instrument; 4) hybridizing the second sequencing primer for segment 2; 5) sequencing the segment 2. For the cases with more than two sequencing primers, step 3-5 can be repeatedly applied for additional sequencing primers.

In some embodiments, the DNA fragment for sequencing is clonally amplified by rolling circle amplification prior to being sequenced.

In some embodiments, the invention's rolony-based sequential sequencing methods are based on the following: Each rolony is a single-stranded DNA concatemer, forms a cluster on the flow cell. It is similar to the Illumina cluster: bridge PCR and NaOH denature to generate single-stranded DNA. One rolony is one cluster. Single-strand DNA (either rolony or Illumina cluster) is the template for SBS sequencing. Rolonies are bound to or seeded on flow cell without grafted oligo (different from Illumina). This is based on electrical charge, which is different from Illumina. Sequencing primers binds to the single-stranded DNA (rolonies) by hydrogen bonds to the complement sequence.

In some embodiments, the invention's sequential sequencing methods (including rolony-based sequential sequencing) includes one or more of the following steps:
a. Binding the first sequencing primers (class A) which i) can be one kind, ii) can be two kinds, for example, pair-end sequencing. Each kind of sequencing primer binds different rolony (cluster), not the same rolony (cluster), or iii) can be multiple kinds, as long as there are no interactions among the primers. Each kind of sequencing primer binds different rolony (cluster), not the same rolony (cluster),
b. Obtaining the first sequencing read (sequencing fragment) by SBS,
c. Removing the first sequencing fragment by the methods described herein (such as denature, degradation, not blocking),
d. Binding the second sequencing primers (class B), which i) can be one kind, ii) can be two kinds, for example, pair-end sequencing. Each kind of sequencing primer bind different rolony (cluster), not the same cluster, or iii) can be multiple kinds, as long as there are no interactions among the primers. Each kind of sequencing primer binds different rolony (cluster), not the same cluster.
e. Obtaining the second sequencing read (sequencing fragment) by SBS.
   In some preferred embodiments, Class A and Class B sequencing primers cannot be applied at the same time on the flow cell.

The invention's sequential sequencing methods can be applied for a) Single-end sequencing on rolonies generated from standard circle, with multiple separate fragments to be sequenced (for example, target sequence, sample index), b) Pair-end sequencing on rolonies generated from dumbbell circle, and c) Pair-end sequencing on rolonies generated from standard circle, but from different strands (top strand, bottom strand) of the library.

A. Rolony Structure for Sequential Sequencing

Certain embodiments pertain to the invention's methods of performing rolling circle amplification (RCA) on polynucleotides to produce concatemers. For example, linear RCA amplifies circular single-stranded DNA by polymerase extension of a complementary primer. This process generates concatemerized copies of the circular DNA template such that multiple copies of a DNA sequence are arranged end to end in a tandem repeat.

There are two exemplary kinds of circle structures which can be a template for RCA to generate rolonies for sequencing, standard circle and dumbbell circle (FIG. 1). In some embodiments, dumbbell structure can be applied for pair-end sequencing. For each kind of circle structure, different sequencing primers are separated in the circle. The corresponding rolony structure with two sequencing primers is shown in FIG. 1. In some embodiments, more than two sequencing primers can be included in a circle structure (U.S. Patent Application No. 62/814,417, incorporated by reference).

Generally, there are three steps for library construction or preparation for rolony based sequencing (FIG. 2). Using standard circle as an example, the details of preparation are listed below.

1) Library Construction.

The library construction includes the following standard process: fragmentation (mechanical or enzymatic process), ends repair, ligation, one or more steps of PCR amplification, one or more steps of cleaning up. However, in order to perform sequential sequencing, the double-stranded final library products should include the schema shown in FIG. 2A.

There are at least 5 regions included in one library fragment for a sequential sequencing. FIG. 2 shows an exemplary embodiment of two sequencing primers. Region 1 and 5 are at the end of the library fragment, with conserved sequence (or named as adaptor), which can be used for bridge oligo hybridization. Region 1, 3, 5 are conserved region, which can be used for RCA amplification primer and sequencing primer design and hybridization. Region 2 and 4 are the sequences to be sequenced, which can be target sequences, index sequences and/or barcode sequences.

In some embodiments, for the case with more than 2 sequencing primers, more fragment/region pair (like regions 3 and 4) can be included before region 5.

In some embodiments, #2 can be the target sequence, always longer than 50nt. #4 can be the region for sample index and/or barcode/umi, always shorter than 25nt. In some embodiments, #2 and #4 can represent vice-versa.

2) Circularization.

The double-stranded DNA template can be denatured by heat or chemical process (like sodium hydroxide (NaOH), but not limited to NaOH). The single-stranded DNA with phosphorylation treatment (either by enzyme, like T4 Polynucleotide Kinase, or being pre-phosphorylated through the last step of PCR amplification during library construction) can be ligated by either single-stranded DNA ligase (e.g. CircLigase ssDNA ligase from Epicentre) or double-stranded DNA ligase (e.g. T4 DNA ligase). When using double-stranded DNA ligase, bride oligo is applied in the process of denature and ligation (FIG. 2D).

3) Amplification by RCA Isothermal Process.

The circled single-stranded DNA of FIG. 2B can be amplified through RCA process by the enzymes with strong strand displacement activities, for example, but not limited to, Phi 29 DNA polymerase, Bst DNA polymerase (large fragment), SensiPhi DNA polymerase (FIG. 2B). The amplified products, rolonies, are the template for sequential sequencing. An example of concatemer unit is listed in FIG. 2C.

B. Sequential Sequencing

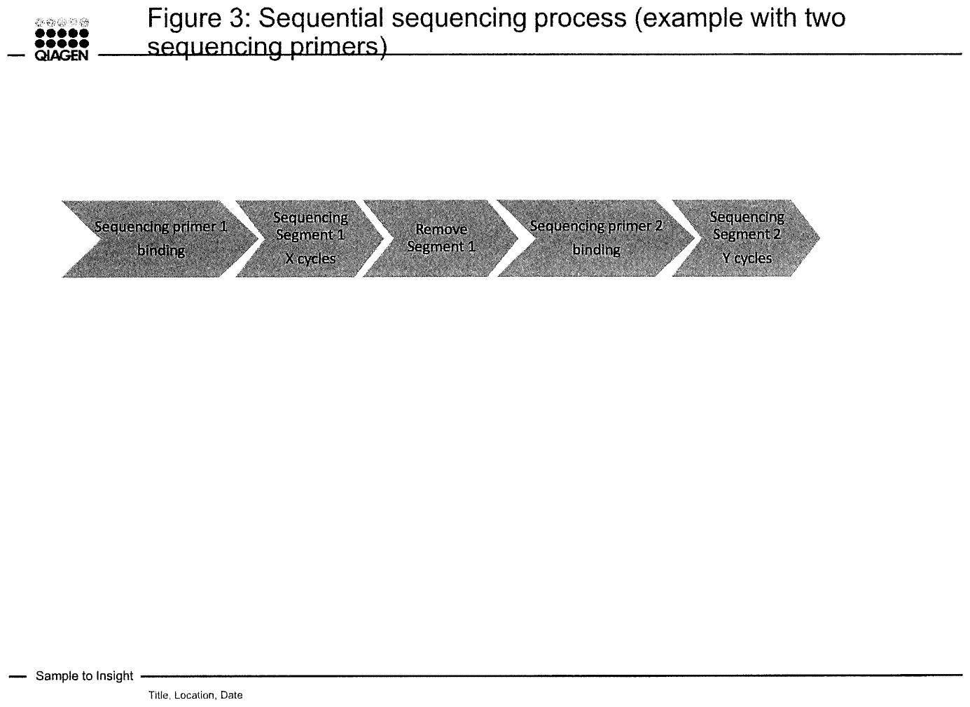

In some embodiments, the rolonies, after seeding on the flow cell, are ready to be sequenced by SBS or other equivalent technologies. FIG. 3 shows a typical workflow for sequential sequencing with two different sequencing primers.

In some embodiments, sequential sequencing includes the following steps for the case with two different sequencing primers:
1) hybridizing the first sequencing primer;
2) sequencing the sequences with X cycles following the first sequencing primer (generating the first sequencing fragment/segment);
3) removing the first sequencing fragment/segment by wash buffer at a certain temperature in the sequencing instrument;
4) hybridizing the second sequencing primer;
5) sequencing the sequences with Y cycles following the second sequencing primer (generating the second sequencing fragment/segment).

In some embodiments, X and Y cycles represent more than 2 cycles in terms of sequencing length for sequencing.

In some embodiments, there are more than two sequencing primers that can be applied to the invention's methods. With this situation, step 3-5 can be repeatedly applied for additional sequencing primers.

In some embodiments, the wash buffer for removing sequencing fragment can be, but not limited to, low ionic strength buffer. With this kind of buffer, the Tₘ of synthesized sequencing fragments can be lower than the sequencer instrument temperature (for example at step 3). Therefore, the synthesized sequencing fragments can be washed off without undesirable impacts on the rolony. The Tₘ can be lower than the instrument temperature for more than 1° C. to ensure removing efficiency. Example of low ionic strength buffer, but not limited to, zero amount or low amount of NaCl (for example, less than or equal to 50 mM), zero amount or low amount of Mg++ buffer. In some embodiments, the wash buffer for removing sequencing fragment can be, but not limited to, denature chemicals, such as sodium hydroxide, formamide, and betaine. Unlike Illumina SBS technology that typically uses sodium hydroxide (NaOH) to generate single-stranded DNA, sodium hydroxide is one of the less desirable choices in the instant invention's rolony-based sequential sequencing platform, and superior results may be obtained by using formamide and/or betaine. With this kind of chemicals, the synthesized sequencing fragments can be denatured and washed off.

In some embodiments, the incubation temperature of sequencing instruments (step 3) can be adjusted to ensure the efficiency of disassociation of previous sequenced fragments.

In some embodiments, the wash buffer for removing sequencing fragment can include accessory proteins/enzymes to increase the efficiency. The proteins can be the enzymes with 3′ to 5′ exonuclease activity or 5′ to 3′ exonuclease activity. Example proteins with 3′ to 5′ exonuclease activity can be, but not limited to, Phi29 DNA polymerase, T4 DNA polymerase, T7 DNA polymerase, Phusion DNA polymerase, Q5 DNA polymerase, and Exonuclease III. Example proteins with 5′ to 3′ exonuclease activity can be, but not limited to, T7 exonuclease, Lambda exonuclease. In some embodiments, engineered proteins with above activities can be applied to the process.

In some embodiments, more than one kind of wash buffers with different components (either low ionic strength buffer, or denature chemicals, or accessory proteins, or combinations) can be applied for the step to remove previous sequenced fragments.

In some embodiments, the first sequencing fragments (X cycles) can be shorter than the second sequencing fragments (Y cycles). For example, the first sequencing fragments can be sample index or barcode. Because Tₘ is always lower with shorter fragments. With this library design, the removing efficiency of the same wash buffer is higher. This design is preferred in some cases.

In some embodiments, the first sequencing fragments (X cycles) can be longer than or equal to the second sequencing fragments (Y cycles). Accessory proteins/enzymes can be applied to degrade the first sequencing fragment to a shorter length which can be efficiently washed off by wash buffer. The accessor proteins can be the enzymes with 3′ to 5′ exonuclease activity or 5′ to 3′ exonuclease activity.

In some embodiments, the sequential sequencing methods described above can be applied to the template other than rolonies. Other clonal amplified products for sequencing can use the methods for sequencing with multiple sequencing primers.

In some embodiments, sequence-by-synthesis (SBS) includes, but not limited to, the following steps for each sequencing cycle: synthesis (or extend), image, cleave, and wash.

In certain embodiments, methods of determining the nucleic acid sequence of one or more clonally amplified concatemers are provided. Determination of the nucleic acid sequence of a clonally amplified concatemer can be performed using variety of sequencing methods known in the art including, but not limited to, sequencing by synthesis (SBS).

Thus, in one embodiment, the invention provides a method for sequential sequencing multiple distinct and separate fragments (such as fragments on a rolony which is a single-stranded DNA template for binding of sequencing primer) in a sequential order on a flow cell surface, comprising: a) hybridizing the first sequencing primer; hybridizing the first sequencing primer; b) sequencing the sequences with X cycles following the first sequencing primer (generating the first sequencing fragment/segment); c) removing the first sequencing fragment/segment (such as by wash buffer at a certain temperature in the sequencing instrument); d) hybridizing the second sequencing primer; e) sequencing the sequences with Y cycles following the second sequencing primer (generating the second sequencing fragment/segment); and f) optionally for additional sequencing primer, repeating the steps c) to e).

In one embodiment, the wash buffer, includes one or more of the following components: a) low ionic strength buffer, b) denature chemicals, and c) accessory proteins with 3′ to 5′ exonuclease activity and/or 5′ to 3′ exonuclease activity.

In a further embodiment, the certain temperature is higher than the melting temperature of the sequencing fragment to be removed in the corresponding wash buffer,

In one embodiment, the rolony can be generated by one of the following kinds of circle templates: a) standard circle, and b) dumbbell circle. In a further embodiment, the rolony can be generated through following process: a) library construction; b) circularization; and c) amplification by RCA isothermal process.

In one embodiment, the sequencing primers can be more than two for different purposes of sequencing, such as for sequencing more than one target sequence.

In one embodiment, the sequential sequencing method does not include any process of synthesis blocking reagents, for example, ddNTP.

In one embodiment, unlike certain prior art methods, the one or more invention's methods described herein lacks (i.e., does not include, and is carried out in the absence of) using a blocking reagent. In one embodiment, the one or more invention's methods described herein lacks (i.e., does not include, and is carried out in the absence of) addition of one or more said blocking reagent to both said first and second reaction mixtures. In one embodiment, the one or more invention's methods described herein lacks (i.e., does not include, and is carried out in the absence of) incorporation of one or more said blocking reagent into any of said first double-stranded DNA sequence produced in step b) and of said second double-stranded DNA sequence produced in step f).

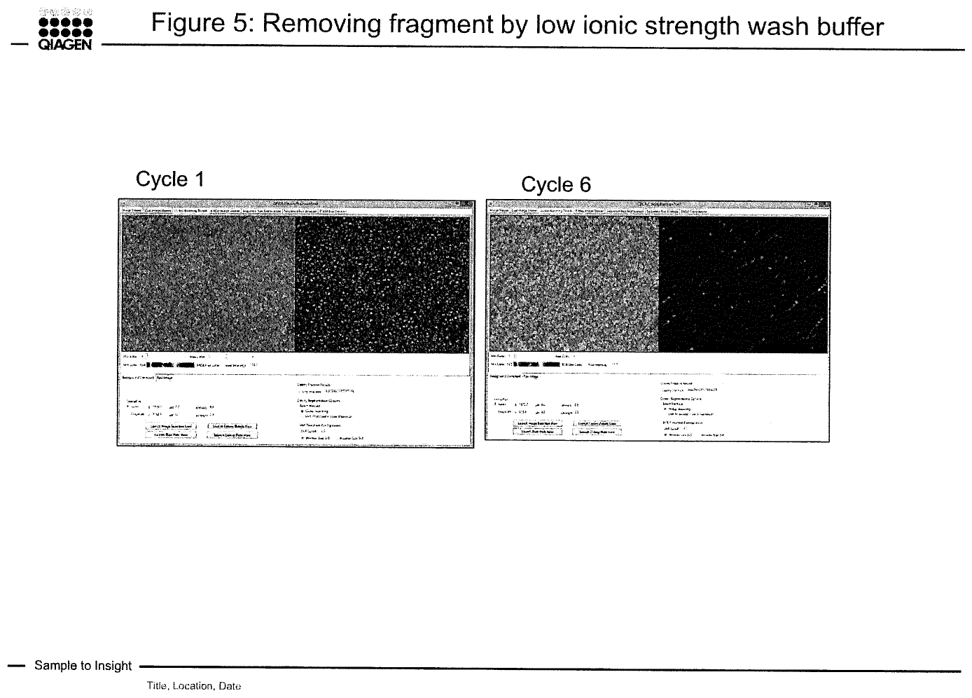

While not intending to limit the removing step to a particular methodology, in one embodiment, the removing of step c) of the one or more invention's methods described herein comprises washing with a buffer having a temperature higher than a melting temperature of said first double-stranded DNA sequence. In one embodiment, the removing of step c) of the one or more invention's methods described herein comprises washing with a buffer having low ionic strength. Example 2 and FIG. 5 show the successful use of an exemplary low ionic strength buffer comprising 50 mM Tris-HCl pH 8.5-8.9 at around 65-70° C., 50 mM sodium chloride, 1 mM EDTA, and 0.05% Tween-20.

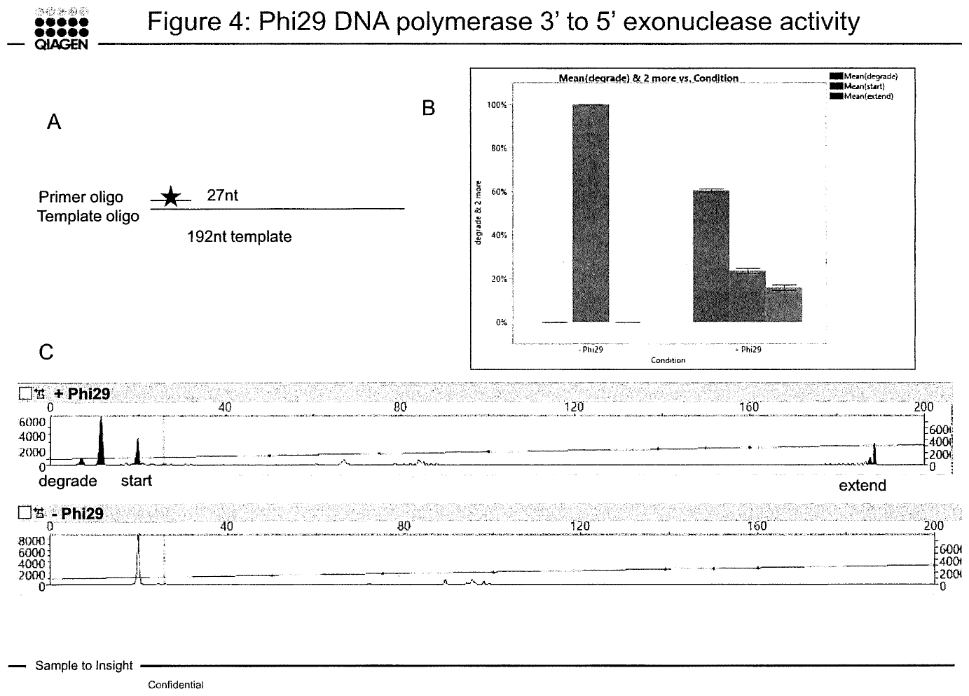

In one embodiment, the removing of step c) of the one or more invention's methods described herein comprises washing with a buffer comprising proteins having 3′ to 5′ exonuclease activity (such as, but not limited to Phi29 DNA polymerase, T4 DNA polymerase, T7 DNA polymerase, Phusion DNA polymerase, Q5 DNA polymerase), Exonuclease III, enzyme having 5′ to 3′ exonuclease activity (such as, but not limited to, T7 exonuclease, Lambda exonuclease, etc.), and compound that denatures double-stranded DNA (such as, but not limited to, denature chemicals, such as sodium hydroxide, formamide, betaine etc.). Examples 1 and 3, and FIG. 4 show the successful use of an exemplary washing buffer comprising the exemplary Phi29 enzyme having 3′ to 5′ exonuclease activity. Example 4 is an example sequential sequencing with the help of exonuclease III. Example 5 is an example sequential sequencing by denature wash buffer.

In one embodiment of the one or more invention's methods described herein, said single-stranded DNA sequence comprises at least a portion of a circular single-stranded DNA, such as rolony. In another embodiment of the one or more invention's methods described herein, said single-stranded DNA sequence is linear. In a further embodiment of the one or more invention's methods described herein, said single-stranded DNA sequence is in contact with (including immobilized on) a flow cell for sequencing.

In one embodiment of the one or more invention's methods described herein, the sequencing of one or both of steps b) and e) comprises sequencing by synthesis (SBS). In one embodiment of the one or more invention's methods described herein, said sequencing steps b) and e) comprises at least two sequencing cycles. In one embodiment of the one or more invention's methods described herein, the number of said sequencing cycles of steps b) and e) is different.

In one embodiment of the one or more invention's methods described herein, said first target sequence is shorter than said second target sequence, and said number of said sequencing cycles (X cycles) of step b) is lower than step e) (Y cycles). For example, the first sequencing fragments of step b) can be sample index or barcode. Because Tₘ is lower with shorter fragments. With this library design, the removing efficiency of the same wash buffer is higher. This design is preferred in some cases.

In one embodiment of the one or more invention's methods described herein, said first target sequence is shorter than said second target sequence, and said number of said sequencing cycles of step b) is higher than step e). This may be desirable in some conditions, such as when the first sequencing fragments of step b) (sequenced with X cycles) is longer than or equal in size to the second sequencing fragments of step e) (sequenced with Y cycles). Accessory proteins/enzymes can be applied to degrade the first sequencing fragment to a shorter length which can be efficiently washed off by wash buffer. The accessory proteins can be the enzymes with 3′ to 5′ exonuclease activity or 5′ to 3′ exonuclease activity. Example 3 shows the exemplary use of 52 cycles (X=52) of standard SBS process for an insert sequence, and of 15 cycles of standard SBS process for an index sequence (Y=15).

In one embodiment of the one or more invention's methods described herein, said first target sequence is with extreme high GC content or long length, accessory proteins/enzymes can be applied to degrade the first sequencing fragment to a shorter length which can be efficiently washed off by wash buffer.

In one embodiment of the one or more invention's methods described herein, the number of said sequencing cycles of steps b) and e) is the same.

### EXPERIMENTAL

The following examples serve to illustrate certain preferred embodiments and aspects of the present invention and are not to be construed as limiting the scope thereof.

### Example 1: Enzymes with the Capability of 3′ to 5′ Exonuclease

Many enzymes have 3′ to 5′ exonuclease activity. For example, the 3′-->5′ exonuclease activity intrinsic to several DNA polymerases plays a primary role in genetic stability; it acts as a first line of defense in correcting DNA polymerase errors. The following experiments were performed to evaluate the enzymes (for example, Phi29 DNA polymerase) and conditions for 3′ to 5′ exonuclease.

Two oligos were designed for this study:

1) Primer oligo (27nt, with internal labeled FAM): /5Phos/AAT GA/iFluorT/ACG GCG ACC ACC GAG ATC TAC

2) Template oligo (192 nt, as a template for primer extension): CA AGC AGA AGA CGG CAT ACG AGA TCG TTA GGA TGT GAC TGG AGT TCA GAC GTG TGC TCT TCC GAT CTA GAT TCT GGC GGG TGC TGA TAG TGT ATC CTA CTA CTT TTG ACT TCT CTG TAG AGG GGA GTC TCA GCT AGA TCG GAA GAG CGT CGT GTA GGG AAA GAG TGT AGA TCT CGG TGG TCG CCG TAT CAT TA

The 600 nM primer oligo was annealed (95° C. for 5 min, 2 min at 60° C. and 5 min at room temperature) with 60 nM template oligo in the 1×Phi29 buffer (50 mM Tris-HCl, 10 mM MgCl2, 10 mM (NH4)2SO4, 4 mM DTT, pH 7.5 @ 25° C.) and 4 mM dNTP in a total volume of 100 uL. 10 units of Phi29 DNA polymerase (Enzymatics, Beverly, Mass.) was added to the tube for 30 min at 30° C. After the reaction, the samples were purified by QIAQuick column with elution in 50 μL TE buffer (QIAGEN, Hilden, Germany). 1 μL of sample was sent out for CE fragment analysis (Genewiz).

The results were shown in FIG. 4. 4A illustrates the oligo structure. 4B shows the percentage of degradation and primer extension. Each condition with or without Phi29 polymerase were tested in triplicates. 4C shows an example figure of with vs. without Phi29 treatment. It demonstrates that the strong 3′ to 5′ exonuclease activity of Phi29 DNA polymerase. However, this polymerase has poor activities of incorporation of ddNTP (data not shown here).

### Example 2: Sequencing Fragment Removal by Low Ionic Wash Buffer

In order to demonstrate the feasibility of removing sequencing fragments by low Tm (or low ionic) wash buffer, the following proof-of-concept experiment was performed.

The tested low ionic strength buffer including the following major components: 50 mM Tris-HCl pH 8.5-8.9 at room temperature, 50 mM sodium chloride, 1 mM EDTA, and 0.05% Tween-20.

Synthetic oligo with 37nt in length (Cy3-GATCTACACTCTTTCCCTACACGACGCTCTTCCGATC), Tm is around 64° C. in above wash buffer, the temperature of the flow cell surface was around 65-70° C. The synthetic oligo was labeled with Cy3 at 5′ end which can be imaged by the SBS sequencer GeneReader 1.8 (development prototype). The image was further analyzed by ImageJ software.

The rolonies were seeded on the surface of flow cell. The synthetic oligo was hybridized on the rolonies in the hybridization buffer. The image was taken at cycle 1. The wash buffer was pumped to the flow cell with the following configuration: 21 μL/sec t at 70° C., total 7004 for each cycle wash. The image was taken after each cycle of wash. Example results were shown in FIG. 5. After 5 cycles of wash, most the fluorescent (Cy3) labeled fragments were gone. It demonstrated the feasibility of removing sequencing fragments from the surface of rolonies by the low ionic strength wash buffer.

### Example 3: Sequential Sequencing with the Help of Enzyme with 3′ to 5′ Exonuclease, Phi29

In order to demonstrate the feasibility of removing sequencing fragment for the 2nd segment sequencing with the enzyme plus low salt wash buffer, the following experiment was performed on GeneReader 1.8 with an automation protocol.

1) The first sequencing primer (GAT+CTA+CAC T+CT T+TC CC+T A+CA CGA CGC TCT TCC GAT C), 37 nt in length, was hybridized with the rolony on flow cell surface (manual process).

2) Sequenced with 52 cycles of standard SBS process for insert sequence (X=52). The total length of sequencing fragment was 89 nt (automation).

3) Then the rolonies were treated with 100 units/mL of Phi29 DNA polymerase at in 1×Phi29 buffer at 30° C. for 60 minutes, and followed by the same wash process disclosed in above example (automation).

4) The second sequencing primer (CGGAAGAGC+ACA+CGT+CTGAACTCCA), 29 nt in length, hybridized with the rolony on the flow cell surface (automation).

5) Sequenced with 15 cycles of standard SBS process for sample index sequence (Y=15) (automation).

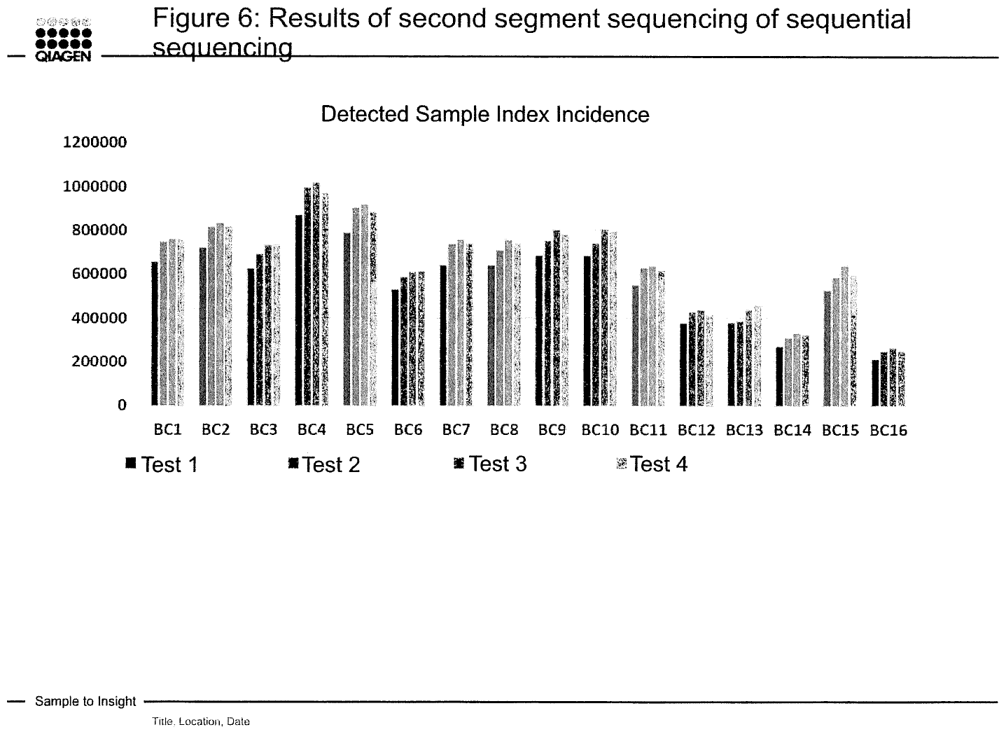

The tests were performed with two runs, each run was with two 33M flow cells. The sequencing results for the second fragment (sample index) are shown in FIG. 6. The index sequences were consistently detected across the four experiments. It demonstrated the feasibility of removing sequence fragments for the 2nd segment sequencing with the help of enzymes which have 3′ to 5′ exonuclease activities.

### Example 4: Sequential Sequencing with the Help of Enzyme with 3′ to 5′ Exonuclease, Exonuclease III

Exonuclease III is a exonuclease which acts by digesting one strand of a dsDNA duplex at a time. In order to demonstrate the feasibility and efficiency of sequential sequencing with the enzyme plus low ionic wash buffer, the following experiment was performed on GeneReader 1.8 with an automation protocol.

1) The first sequencing primer (GAT+CTA+CAC T+CT T+TC CC+T A+CA CGA CGC TCT TCC GAT C), 37 nt in length, was hybridized with the rolony on 33M flow cell surface (manual process).

2) Sequenced with 51 cycles of standard SBS process for insert sequence (X=51). The total length of sequencing fragment was 88 nt (automation).

1) Then the rolonies were treated with 100 units/mL of Exo III enzyme (Enzymatics, Beverly, Mass.) in 1× reaction buffer provided by supplier at 37° C. for 60 minutes, and followed by the same wash buffer disclosed in above example at 70° C., with the following procedure: 2 rounds of washing with 700 μL buffer at 214/sec, incubation at 70° C. for 3 minutes, and another 3 rounds of washing with 700 μL buffer at 214/sec (automation).

3) The second sequencing primer (CGGAAGAGC+ACA+CGT+CTGAACTCCA), 29 nt in length, hybridized with the rolony on the flow cell surface (automation).

4) Sequenced with 15 cycles of standard SBS process for sample index sequence (Y=15) (automation).

The data were processed to get the raw reads and mapped reads, with and without demultiplexing. The quantitative evaluation matrix of % index, % aligned and % Q-score ratio were calculated and listed in Table 1. It demonstrated the efficiency of sequential sequencing with the help of enzymes which have 3′ to 5′ exonuclease activities. This method is not dependent on the sequence GC content and segment length. It can be applied for the cases of high GC content sequences and/or long reads.

### Example 5: Sequential Sequencing with the Denature Buffer

Formamide and Q-solution (QIAGEN, Hilden, Germany) are typical PCR additives to reduce the buffer Tm. In order to demonstrate the feasibility and efficiency of sequential sequencing with the denature buffer, the following experiment was performed on GeneReader 1.8 with an automation protocol. Two kinds of denature buffer were evaluated. Buffer 1: 40 mM Tris-HCl, pH 8.5 at room temperature, with 10% formamide. Buffer 2: 25 mM Tris-HCl, pH 8.5 at room temperature, with 50% Q-solution.

1) The first sequencing primer (GAT+CTA+CAC T+CT T+TC CC+T A+CA CGA CGC TCT TCC GAT C), 37 nt in length, was hybridized with the rolony on 33M flow cell surface (manual process).

2) Sequenced with 51 cycles of standard SBS process for insert sequence (X=51). The total length of sequencing fragment was 88 nt (automation).

3) Then the rolonies were treated with either Buffer 1 or Buffer 2 with the following protocol: 2 round of wash with 7004 buffer at 80° C. at 214/sec, incubation at 80° C. for 3 minutes, and another 3 rounds of wash with 7004 buffer at 80° C. at 21 μl/sec (automation).

4) The second sequencing primer (CGGAAGAGC+ACA+CGT+CTGAACTCCA), 29 nt in length, hybridized with the rolony on the flow cell surface (automation).

5) Sequenced with 15 cycles of standard SBS process for index sequence (Y=15) (automation).

The data were processed to get the raw reads and mapped reads, with and without demultiplex process. The quantitative evaluation matrix of % index, % aligned and % Q-score ratio were calculated. The results are listed in Table 1. It demonstrated the efficiency of removing sequence fragments with the denature conditions. This method is fast and cost-efficient, suitable for short reads sequence without extremely high GC contents.

TABLE 1
Evaluation matrix for sequential sequencing.

| Method | % index | % Aligned | Q-Score Ratio |
|---|---|---|---|
| Degradation (Exo III) | 64.8% | 85.1% | 84.6% |
| Denature (Formamide) | 61.3% | 88.8% | 93.5% |
| Denature (Q-solution) | 81.9% | 93.1% | 98.1% |

Each and every publication and patent mentioned in the above specification is herein incorporated by reference in its entirety for all purposes. Various modifications and variations of the described methods and system of the invention will be apparent to those skilled in the art without departing from the scope and spirit of the invention. Although the invention has been described in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. A method for sequentially sequencing a plurality of target sequences, said method comprising
i) providing
a) a single-stranded DNA sequence comprising a plurality of target sequences,
wherein each member of said plurality of target sequences is operably fused to an oligonucleotide sequence, and
wherein the sequence of said oligonucleotide sequence fused to each said member of said plurality of target sequences is different from the sequence of said oligonucleotide sequence fused to each of the other members of said plurality of target sequences, and
b) a plurality of sequencing primers, wherein each member of said plurality of sequencing primers is complementary to and specifically binds with a different said oligonucleotide sequence,
ii) sequentially sequencing two or more said member of said plurality of target sequences, said sequentially sequencing comprises
a) hybridizing one or more first member of said plurality of sequencing primers to its said complementary oligonucleotide sequence in a first reaction mixture to produce a first hybridized sequencing primer,
b) extending said first hybridized sequencing primer to produce a first sequencing read, said extending comprises producing a first double-stranded DNA sequence containing a complement of a first said member of said plurality of target sequences operably fused to said first member of said plurality of sequencing primers, and comprises sequencing at least one strand of said first double-stranded DNA sequence,
c) removing the extended sequence produced in step b) from said first reaction mixture,
d) hybridizing one or more second member of said plurality of sequencing primers to its said complementary oligonucleotide sequence in a second reaction mixture to produce a second hybridized sequencing primer, and
e) extending said second hybridized sequencing primer to produce a second sequencing read, said extending comprises producing a second double-stranded DNA sequence containing a complement of a second said member of said plurality of target sequences operably fused to said one or more second member of said plurality of sequencing primers, and comprises sequencing at least one strand of said second double-stranded DNA sequence.

2. The method of claim 1, further comprising repeating steps c) to e) to sequence members of said plurality of target sequences that are different from both said one or more first member of said target sequence and said one or more second member of said target sequence.

3. The method of claim 1, wherein said method lacks using a blocking reagent.

4. The method of claim 3, wherein said method lacks addition of one or more said blocking reagent to both said first and second reaction mixtures.

5. The method of claim 3, wherein said method lacks incorporation of one or more said blocking reagent into any of said first double-stranded DNA sequence produced in step b) and of said second double-stranded DNA sequence produced in step e).

6. The method of claim 1, wherein said removing of step c) comprises washing with a buffer having a temperature higher than a melting temperature of said first double-stranded DNA sequence produced in step b).

7. The method of claim 1, wherein said removing of step c) comprises washing with a buffer having low ionic strength.

8. The method of claim 1, wherein said removing of step c) comprises washing with a buffer comprising one or more of a protein having 3′ to 5′ exonuclease activity, and an enzyme having 5′ to 3′ exonuclease activity.

9. The method of claim 1, wherein said removing of step c) comprises washing with a buffer comprising a compound that denature DNA, or reducing melting temperature of double-stranded DNA.

10. The method of claim 1, wherein said single-stranded DNA sequence comprises at least a portion of a rolony.

11. The method of claim 1, wherein said single-stranded DNA sequence is linear.

12. The method of claim 1, wherein said sequencing steps b) and e) comprise at least two sequencing cycles.

13. The method of claim 1, wherein the number of said sequencing cycles of steps b) and e) is different.

14. The method of claim 1, wherein said first target sequence is shorter than said second target sequence, and said number of said sequencing cycles of step b) is less than step e).

15. The method of claim 1, wherein said first target sequence is longer than said second target sequence, and said number of said sequencing cycles of step b) is more than step e).

16. The method of claim 1, wherein the number of said sequencing cycles of steps b) and e) is the same.

17. The method of claim 1, wherein said hybridizing said one or more first member of said plurality of sequencing primers of step ii) a) is in the absence of said hybridizing said one or more second member of said plurality of sequencing primers of said step ii) d).

18. The method of claim 1, wherein said hybridizing two or more said first member of said plurality of sequencing primers of step ii) a) is substantially at the same time.

19. The method of claim 1, wherein said hybridizing of two or more of said second member of said plurality of sequencing primers of step ii) d) is substantially at the same time.

20. The method of claim 10, wherein said rolony is generated from a DNA template comprising one or both of standard circle and dumbbell circle.

21. The method of claim 1, comprising hybridizing, for each sequencing event, at least one of said plurality of sequencing primers to said member of said plurality of target sequences to start a sequencing read.

22. The method of claim 1, said sequentially sequencing said plurality of target sequences comprises one more of
i) single-end sequencing on rolonies generated from a standard circle, said rolonies comprising multiple separate fragments to be sequenced,
ii) pair-end sequencing on rolonies generated from a dumbbell circle, and
iii) pair-end sequencing on rolonies generated from a standard circle of a library, said different strands comprising a top strand and a bottom strand.

23. A kit for sequentially sequencing single-stranded DNA sequence that comprises a plurality of target sequences, wherein each member of said plurality of target sequences is operably fused to an oligonucleotide sequence, said kit comprising
a) a plurality of sequencing primers, wherein each member of said plurality of sequencing primers is complementary to and specifically binds with a different segment of said oligonucleotide sequence,
b) a reagent for removing sequencing fragments, and
c) instructions for using said plurality of sequencing primers and said reagent.

24. The kit of claim 23, further comprising one or more of d) a reagent for denaturing sequencing fragments, e) a reagent for degradation of sequencing fragments, f) a reagent for removing sequencing primers that do not specifically bind with said different segment of said oligonucleotide sequence, g) a reagent for removing said denaturing reagent, and h) a reagent for removing said degradation reagent.
